(19) United States
(12) Patent Application Publication
(10) Pub. No.: US 2015/0305688 A1
(43) Pub. Date: Oct. 29, 2015

(54) METHOD OF DETERMINING DISCHARGE READINESS CONDITION FOR A PATIENT AND SYSTEM THEREOF

(71) Applicant: Wipro Limited, Bangalore (IN)

(72) Inventors: Satish Prasad Rath, Bangalore (IN); Upendra Suddamalla, Kadiri (IN); Vinod Pathangay, Bangalore (IN)

(21) Appl. No.: 14/299,906

(22) Filed: Jun. 9, 2014

(30) Foreign Application Priority Data
Apr. 25, 2014 (IN) 2092/CHE/2014

(51) Int. Cl. A61B 5/00; A61B 5/16; A61B 5/0402; A61B 5/0205; A61B 5/11

(52) U.S. Cl. CPC A61B 5/7282; A61B 5/02055; A61B 5/1118; A61B 5/0402; A61B 5/4848; A61B 5/4866; A61B 5/4824; A61B 5/165; A61B 5/742; A61B 5/486; A61B 5/01

(57) ABSTRACT

Embodiments of the present disclosure provide a method of determining discharge readiness condition for a patient. The sensors are placed on patient to monitor the physiological condition of the patient and to transmit physiological data to computing device. The patient also provides non-physiological data using mobile device to computing device. The processor configured in computing unit assigns predefined weighted ratio to each of the physiological data and non-physiological data to generate weighted physiological and non-physiological data. The processor generates recovery score using the weighted physiological data and the non-physiological data and compares the recovery score with reference recovery score. The reference recovery score is personalized for each patient using weighted personal reference score and weighted EMR reference score. If recovery score exceeds reference recovery score then patient is ready for discharge else the patient is not ready to be discharged and hence appropriate suggestions are provided to improve recovery score.

This application claims the benefit of Indian Patent Application Serial No. 2092/CHE/2014, filed Apr. 25, 2014, which is hereby incorporated by reference in its entirety.

### FIELD OF THE INVENTION

The present disclosure relates to monitoring health condition of a patient. In particular, the present disclosure relates to a method of determining discharge readiness condition for a patient based on the health condition.

### BACKGROUND OF THE INVENTION

The discharge of a patient from a hospital depends on recovery of the patient from an illness/ailment. At present, the process of determining when exactly the patients are ready to be discharged has traditionally been a subjective decision by the physicians and the hospital professionals. Due to the highly subjective nature of this decision, there is a great deal of variability in determining when the patients are ready for discharge. The longer stay of the patient in the hospital leads to many adverse clinical and financial outcomes due to the subjective and intermittent nature of evaluation of the patient's recovery of health. Some of the adverse clinical and financial outcomes caused due to incorrect assessment of the discharge condition of the patient are late discharge from the hospital and early discharge from the hospital.

The problems associated with late discharge of the patient from the hospital is mismanagement and wastage of the healthcare resources due to longer stay of the patient who is ready to be discharged, anxiety and discomfort in patients and attendants with limited resources, decreased availability of healthcare services for the patients who are in need of the healthcare resources etc. Generally, hospitals have limited beds in the Intensive Care Unit (ICU) as they require huge investment and for patients, longer duration of hospitalization and ICU care are involved with high costs and discomfort due to the ICU environment.

The early discharge of the patient before the patient is stable enough for less intensive monitoring and care, are at risk for complications. The complications such as readmission of the patient with the same or related ailment resulting in increased stress placed on patient and patient's families. In addition to the increased stress placed on patient and patient's families, patients readmitted tend to have higher risk of mortality.

In an effort to overcome the aforementioned problems various systems and methods have been proposed. One of such methods predicts the discharge recommendations based on the predicted change in the patient record for all possible medical treatments. Another method adaptively modifies the discharge criteria/guidelines for patients based on patient conditions in the hospital. The discharge condition is modified such that the patients who are wrongly discharged or wrongly not-discharged are minimized. Another method uses two predictive models to calculate risk of death and risk of readmission. Further it calculates risk of discharge from the above two risks. The described existing methods, however, do not disclose a method of assessing discharge readiness of the patient based on recovery of the patient, wherein recovery of the patient is compared with a personalized discharge condition of the patient. Further, none of the described existing methods discloses a method of tracking the recovery of the patient after the patient has been discharged from the hospital. Also, the prior-art references do not disclose a method of providing suggestions to the patients to improve the recovery.

Accordingly, a need exists for a method which provides a means to assess discharge readiness of the patient by continuous monitoring of recovery of the patient and providing suggestions to the patient to improve the recovery.

### SUMMARY OF THE INVENTION

One or more shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

The present disclosure provides method for determining discharge readiness condition for a patient. The method comprising receiving, by a computing unit, at least one of one or more physiological data from plurality of sensors placed on the patient and one or more non-physiological data from a mobile device associated to the patient. Thereafter, the computing unit assigns a first weighted ratio to each of the physiological data received from the plurality of sensors and the one or more non-physiological data received from the mobile device to generate a weighted physiological data and non-physiological data. The computing unit generates a recovery score using the weighted physiological data and the weighted non-physiological data. Thereafter, the computing unit determines the discharge readiness condition of the patient when the recovery score exceeds a reference recovery score, wherein the reference recovery score is generated using a weighted personal reference score of the patient and a weighted Electronic Medical Record (EMR) reference score.

Further, the present disclosure provides a computing unit for determining discharge readiness condition for a patient. The computing unit comprises at least one processor and a memory storing instructions executable by the at least one processor, wherein the instructions configure the at least one processor to receive at least one of one or more physiological data from plurality of sensors placed on the patient and one or more non-physiological data from a mobile device associated to the patient. The processor is further configured to assign a first weighted ratio to each of the physiological data received from the plurality of sensors and the non-physiological data received from the mobile device to generate a weighted physiological data and a weighted non-physiological data. The processor is furthermore configured to generate a recovery score using the weighted physiological data and the weighted non-physiological data, and determine the discharge readiness condition of the patient when the recovery score exceeds a reference recovery score. In one embodiment, the reference recovery score is generated using a weighted personal reference score of the patient and a weighted Electronic Medical Record (EMR) reference score.

Furthermore, the present disclosure provides a non-transitory computer readable medium including operations stored thereon that when processed by at least one processor cause a system to perform the acts of receiving at least one of one or more physiological data from plurality of sensors placed on the patient and one or more non-physiological data from a mobile device associated to the patient. The processor further cause the system to perform the act of assigning a weighted ratio to each of the physiological data received from the plurality of sensors and the non-physiological data received from the mobile device to generate a weighted physiological data and a weighted non-physiological data. Further, the processor causes the system to perform the acts of generating a recovery score using the weighted physiological data and the weighted non-physiological data and determining the discharge readiness condition of the patient when the recovery score exceeds a reference recovery score, wherein the reference recovery score is generated using a weighted personal reference score of the patient and a weighted Electronic Medical Record (EMR) reference score.

The foregoing summary is illustrative only and is not intended to be in any way limiting. In addition to the illustrative aspects and features described above, further aspects, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 illustrates an exemplary environment for determining discharge readiness condition for a patient in accordance with some embodiments of the present disclosure;

FIG. 2 illustrates an exemplary block diagram showing process of determining a discharge readiness condition for a patient in accordance with some embodiments of the present disclosure;

FIG. 3 illustrates graphical representation of recovery of patient on the display unit in accordance with an exemplary embodiment of the present disclosure;

FIG. 4 shows a flowchart illustrating an exemplary process for determining reference recovery score of the patient in accordance with some embodiments of the present disclosure; and

FIG. 5 shows a flowchart illustrating a method for determining discharge readiness condition for the patient in accordance with some embodiments of the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION OF THE INVENTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific aspect disclosed may be readily utilized as a basis for modifying or designing other structures for carrying out the same purposes of the present disclosure.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or apparatus.

As used herein, the term “patient” refers to any recipient of healthcare services. For the purpose of this disclosure, the term “physiological data” refers to data associated with physiological parameters of the patient. The physiological parameters include, but not limited to, body temperature, heart rate, body acceleration and respiration rate. For the purpose of this disclosure, the term “non-physiological data” refers to data associated with non-physiological parameters of the patient. The non-physiological parameters may include, but not limited to, food intake, medicine intake and discomfort level of the patient.

Embodiments of the present disclosure relate to a method for determining a discharge readiness condition for a patient. More particularly, a method of determining discharge readiness condition for a patient based on at least one of one or more physiological data and one or more non-physiological data received from a patient is disclosed. The method includes receiving by a computing unit at least one of one or more physiological data from plurality of sensors placed on the patient and one or more non-physiological data from a mobile device associated to the patient. For each of the received physiological data and non-physiological data, a first weighted ratio is assigned to generate a weighted physiological data and a weighted non-physiological data. Thereafter, a recovery score is generated using the weighed physiological data and the weighted non-physiological data. The generated recovery score is compared with a reference recovery score which is personalized for each patient. The reference recovery score is generated using a weighted personal reference score of the patient and a weighted Electronic Medical Record (EMR) reference score. When the recovery score exceeds the reference score, the discharge readiness condition for the patient is determined. If the generated recovery score is less than the reference recovery score, then the patient is not ready to be discharged and appropriate suggestions are provided to the patient to improve the recovery score. Even after the patient is discharged, the patient's recovery score is continuously monitored to provide suggestions to the patient to cure the illness.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

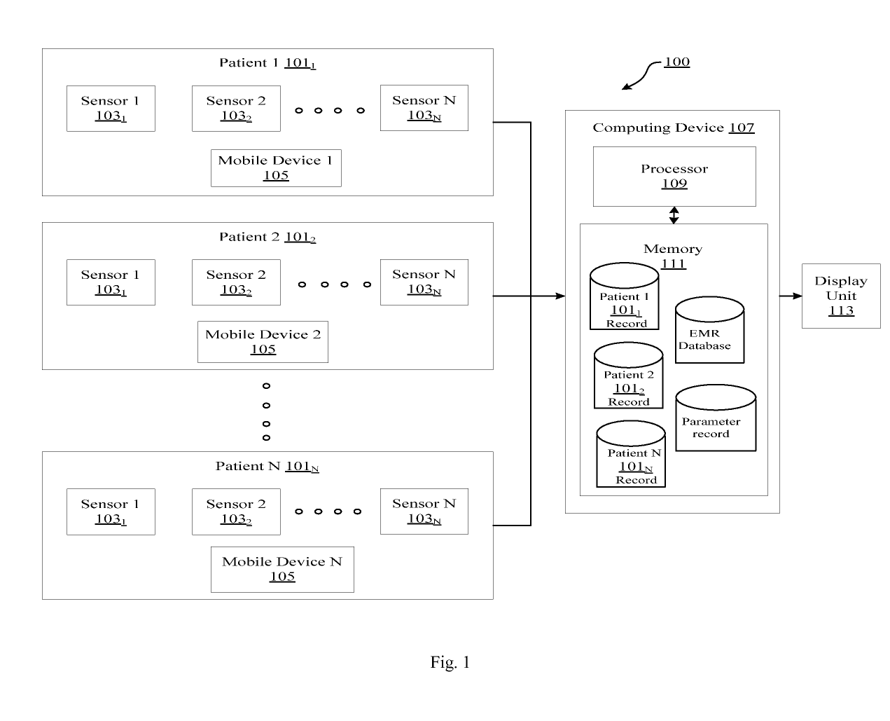

FIG. 1 illustrates an exemplary environment 100 for determining discharge readiness condition for a patient in accordance with some embodiments of the present disclosure.

As shown in FIG. 1, the environment 100 includes a plurality of patients, patient 1 1011 to patient N 101N (collectively referred to as plurality of patients 101), a computing device 107 and a display unit 113 associated with the computing device 107. The environment also illustrates one or more sensors, sensor 1 1031 to sensor N 103N (collectively referred as one or more sensors 103) are placed on the body of the patient to monitor the physiological condition of the patient and to transmit the physiological data to the computing device 107. Examples of the one or more sensors 103 include, but are not limited to physiological sensor, activity sensor, motion sensor and emotion sensor. The one or more sensors 103 provide physiological data to the computing device 107. The one or more sensors 103 to be placed on the patient are based on the type of illness/ailment being suffered by the patient. For example, one or more sensors placed on the patient if the patient has fever are physiological sensor [temperature sensor] and activity sensor. Similarly, if the patient is suffering from respiratory problems, the one or more sensors placed on the patient are physiological sensor [respiratory rate sensor] and activity sensor. Each patient may be associated with a mobile device 105. Example of the mobile device includes, but not limited to, a mobile phone, a tablet and a computer. The mobile device 105 may be used by the patient, patient's guardians and/or patient's care takers. The mobile device 105 may include one or more applications using which the patient may provide one or more non-physiological data associated with the illness. As shown in the FIG. 1, one or more sensors and mobile devices 105 are communicatively coupled to the computing device 107 through a network (not shown) for facilitating transmission of the data to the computing device.

The network 106 may be a wireless network, wired network or a combination thereof. The network 106 can be implemented as one of the different types of networks, such as intranet, local area network (LAN), wide area network (WAN), the internet, and such. The network 106 may either be a dedicated network or a shared network, which represents an association of the different types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the network 106 may include a variety of network devices, including routers, bridges, servers, computing devices, storage devices, etc.

The computing unit 107 includes at least one processor 109 and a memory 111 for storing instructions executable by the at least one processor 109. The processor 109 may comprise at least one data processor for executing program components and for executing user or system generated requests. A user may include a person, a person using a device such as those included in this disclosure, or such a device itself. The processor 109 may include specialized processing units such as integrated system (bus) controllers, memory management control units, floating point units, graphics processing units, digital signal processing units, etc. The processor 109 may include a microprocessor, such as AMD Athlon, Duron or Opteron, ARM's application, embedded or secure processors, IBM PowerPC, Intel's Core, Itanium, Xeon, Celeron or other line of processors, etc. The processor 109 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc. Among other capabilities, the processor 109 is configured to fetch and execute computer-readable instructions stored in the memory 111. The memory 111 can include any non-transitory computer-readable medium known in the art including, for example, volatile memory (e.g., RAM), and/or non-volatile memory (e.g., EPROM, flash memory, etc.).

The computing unit 107 receives the one or more physiological data from the one or more sensors 103 and one or more non-physiological data from the mobile device 105 associated with each patient. The computing unit 107 includes the memory 111 to store the received data from the plurality of patients 101. The data received from each patient 101 is stored separately in each database configured in the memory 111. For example, the data received from patient 1 is stored in the patient 1 1011 record. Similarly, the data received from patient 2 1012 is stored in the patient 2 1012 record and the data received from the patient N 101N is stored in the patient N 101N record. The memory also comprises one or more databases such as EMR database to store the generalized EMR reference scores and parameter record database to store one or more parameters associated with each type of illness.

In operation, the computing unit 107 receives the one or more physiological data from the one or more sensors 103 placed on the patient and the one or more non-physiological data from the mobile device 105 associated with the patient. The processor 109 dynamically assigns a first weighted ratio to each of physiological data and the non-physiological data. The first weighted ratio is a set of predefined weights or a scaling factor. The predefined weight is multiplied with the data to obtain a final weighted value. In some embodiments, the set of predefined weights may be between 0 and 1. The processor 109 normalizes the set of predefined weights assigned to the data such that, the sum of all the predefined weights is equal to one. In some other embodiments, the set of predefined weights may be represented in terms of percentage value wherein the processor 109 normalizes the set of predefined weights assigned to the data such that, the sum of all the weights is equal to 100 percent. The processor 109 generates the recovery score for the patient by adding all the weighted physiological data and non-physiological data. The generated recovery score of each patient is stored in their respective patient records. The processor 109 compares the generated recovery score with a reference recovery score to identify whether the patient is ready for discharge from the hospital. The reference recovery score is personalized for each patient. The reference recovery score is generated using a weighted personal reference score and a weighted Electronic Medical Record (EMR) reference score. The weighted EMR reference score is determined by assigning a second weighted ratio to the EMR reference score. The second weighted ratio is a predefined weight or a scaling factor assigned to the EMR reference score. The predefined weight is multiplied with the EMR reference score to obtain the weighted EMR reference score. In some embodiments, the predefined weight may be between 0 and 1. In some other embodiments, the predefined weight may be represented in terms of percentage value. The weighted EMR reference score is stored in EMR database configured in the memory 111. The EMR reference score is a generalized reference score for a group of patients with similar age and gender. For each group of patients, the processor 109 determines a different EMR reference score. The second weighted ratio is dynamically assigned to the EMR reference score.

The personal reference score is determined by assigning a third weighted ratio to each of the physiological data and the non-physiological data of the patient recorded during normal health condition either by placing one or more sensors on the patient or from the one or more previous health reports of the patient.

In some exemplary embodiments, one or more sensors 103 may be placed on the person during normal health condition of the person to record the physiological data of the person. Similarly, to record the non-physiological data of the person during normal health condition the person has to provide the non-physiological data using the mobile device 105.

In some other exemplary embodiments, the previous health reports of the person may be used to determine the personal reference score of the patient.

The processor 109 dynamically assigns a third weighted ratio to the personal reference score of the person to generate a weighted personal reference score. The third weighted ratio is a predefined weighted or a scaling factor assigned to the personal reference score. The predefined weight is multiplied with the personal reference score to obtain the weighted personal reference score. In some embodiments, the predefined weight may be between 0 and 1. In some other embodiments, the predefined weight may be represented in terms of percentage value.

In some embodiments, if the predefined weights i.e the second weighted ratio and the third weighted ratio are in between 0 and 1 then the processor 109 normalizes the predefined weights such that the sum of the second weighted ratio and the third weighted ratio equals to one. In some other embodiments, if the predefined weights i.e the second weighted ratio and the third weighted ratio are represented in terms of percentage values, then the processor 109 normalizes the predefined weights such that the sum of the second weighted ratio and the third weighted ratio equals to 100 percent.

The reference recovery score is computed by adding the weighted personal reference score and the weighted EMR reference score.

Upon determining the reference recovery score for the patient for a particular illness, the processor 109 compares the recovery score with the reference recovery score. If the recovery score exceeds the reference recovery score, the processor 109 determines that the patient is ready for discharge from the hospital. If the recovery score is less than the reference recovery score, then the processor 109 determines that the patient is still not ready to be discharged from the hospital. If the recovery score is less than the reference recovery score, then the processor 109 provides appropriate suggestions to the patient to improve the recovery score. In some embodiments, the recovery score may be less because of improper intake of food. Therefore, the suggestions provided to improve the recovery score will be based on the food intake parameter of the patient. Similarly, the recovery score patient may be less due to less activity level when compared with the targeted activity level for the patient. In such scenario, the suggestions provided by the computing unit to the patient would be to walk around for 5-10 minutes daily.

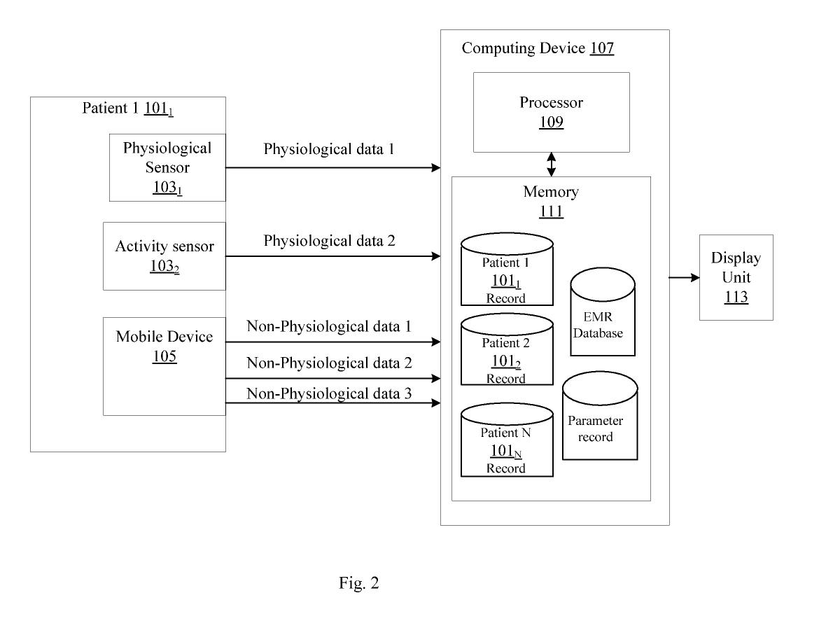

FIG. 2 illustrates an exemplary process of determining a discharge readiness condition for a patient in accordance with some embodiments of the present disclosure. In an exemplary embodiment, the patient 1 1011 may be suffering from fever. In some embodiments, the computing unit 107 stores a parameter record in the memory 111 as shown in the below Table 1. The parameter record indicates the one or more parameters to be monitored for different type of illness.

TABLE 1

| | Parameters | | | | | |
|---|---|---|---|---|---|---|
| | | | | | | Parameter N |
| | | | | Parameter 4 | - | Blood |
| | Parameter 1 | Parameter 2 | Parameter 3 | Respiration | - | sugar |
| Illness | Temperature | Activity level | Cardiac event | Rate Analysis | - | Level |
| Illness 1-Fever | Yes | Yes | No | No | - | No |
| | | | | | - | |
| | | | | | - | |
| Illness 2- | No | Yes | No | No | - | No |
| Hepatitis B | | | | | - | |
| | | | | | - | |
| Illness 3- | No | No | No | Yes | - | No |
| Pneumonia | | | | | - | |
| | | | | | - | |
| ! | ! | ! | ! | ! | ! | ! |
| ! | ! | ! | ! | ! | ! | ! |
| ! | ! | ! | ! | ! | ! | ! |
| Illness 4- | No | Yes | No | Yes | | No |
| Asthma | | | | | | |

In the exemplary embodiment, the patient 1 1011 is being suffered from fever. Therefore, the parameters to be monitored are body temperature and the activity level of the patient. Accordingly, the one or more sensors 103 to be placed on the patient 1 1011 are physiological sensor and the activity sensor. The physiological sensor [temperature sensor] measures the body temperature of the patient and provides the physiological data 1 associated with the body temperature of the patient to the computing device 107. The computing unit 107 generates a recovery score based on the physiological data 1. If the body temperature of the patient is more, then the corresponding recovery score generated is less.

The activity sensor measures the activity level of the patient 1 1011 i.e whether the patient is able to walk freely or if there are any disturbances for the patient during sleep etc. and provides the physiological data 2 associated with the activity level of the patient 1 1011 to the computing device 107. The computing device 107 generates a recovery score based on the physiological data 2. If the patient is able to walk freely then the recovery score generated is more but if the patient 1 1011 is lying on a bed for more time then the recovery score generated is less. The patient 1 1011 also provides one or more non-physiological data during the illness to the computing device 107 using the mobile device 105. The patient 1 1011 manually provides the food intake data referred as non-physiological data 1, medicine intake data referred as non-physiological data 2 and discomfort level data referred as the non-physiological data 3 to the computing device 107 through the mobile device 105. The computing device 107 generates a recovery score for each of the non-physiological data. If the patient 1 1011 has skipped the medicines then the recovery score generated will be less. If the patient's 1 1011 food intake is proper then the recovery score generated will be more and if there is any discomfort or pain to the patient 1 1011 then the generated recovery score will be less.

The patient 1 1011 is monitored continuously for over the period of time, for example, five days from the start of observation. The Table 2 below provides details of the recovery score associated with the patient 1 1011 on daily basis.

TABLE 2

| | Recovery | Recovery | Recovery | Recovery | Recovery | |
|---|---|---|---|---|---|---|
| No. of days | Score of | Score of | Score of | Score of | Score of | |
| from start | Parameter | Parameter | Parameter | Parameter | Parameter | |
| of | 1-body | 2-activity | 3-food | 4-Medicine | 5-Discomfort | Recovery |
| observation | temperature | level | intake | intake | level | Score |
| Day 1 | 0 | 0 | 1 | 1 | 1 | 3 |
| Day 2 | 1 | 1 | 1 | 1 | 1 | 5 |
| Day 3 | 2 | 2 | 2 | 2 | 2 | 10 |
| Day 4 | 2 | 2 | 2 | 2 | 2 | 10 |
| Day 5 | 3 | 3 | 2 | 2 | 2 | 12 |

As seen from the above Table 2, on day 1, the recovery score of the patient is 3 by adding the recovery score of each parameter associated with the illness of the patient 1 1011. Similarly, on day 2, the recovery score is 5, on day 3 the recovery score is 10, on day 4 the recovery score is 11 and on day 5 the recovery score is 12. The processor 109 compares the generated recovery score with the reference recovery score of the patient. The reference recovery score of the patient 1 1011 is personalized using the weighted personal reference score of the patient and the weighted EMR reference score. The processor 109 retrieves the EMR reference score from the EMR database stored in the memory 111. The EMR reference score is a generalized reference score for a group of patients of similar age and gender. The EMR reference score is determined for each kind of illness and stored in the EMR database. In an exemplary embodiment, the EMR reference score for the illness associated with the patient 1 1011 is 12. The second weighted ratio Wt 1 assigned to the EMR reference score is 0.5.

The personal reference score of the patient 1 1011 is determined by at least one of the physiological data and one or more non-physiological data recorded during normal health condition of the patient 1 1011 and previous health reports of the patient 1 1011.

In some embodiments, the one or more sensors 103 are placed on the person during normal health condition of the person to determine the physiological data of the person. The person also provides one or more non-physiological data during the normal health condition. The processor 109 generates a reference score for each of the received physiological data and non-physiological data. Thereafter, the processor 109 computes the personal reference score using each of the reference score. The processor 109 assigns a third weighted ratio to the personal reference score. In some other embodiments, the physicians may generate a personal reference score using the previous health reports of the patient. The health check-up reports may provide information related to fitness of the person. The processor 109 generates a reference score for each of the parameter indicated in the report and thereafter computes the personal reference score using the reference score of each parameter required to assess illness of the patient 1 1011. In an exemplary embodiment, the personal reference score of the person during normal health condition is 10. The third weighted ratio Wt 2 assigned to the personal reference score is 0.5.

In some embodiments, the reference recovery score is calculated using the below formula.

Reference recovery score=Wt1*EMR reference score+Wt2*personal reference score

Reference recovery score=0.5*12+0.5*10=11

The processor 109 compares the generated recovery score with the reference recovery score. The generated recovery score is 12 and the reference recovery score is 11. Since the generated recovery score exceeds the reference recovery score, the computing unit 107 determines that the patient 1 1011 is ready for discharge from the hospital.

In some embodiments, if the generated recovery score is less than the reference recovery score then the computing unit 107 identifies whether the patient 1 1011 is at hospital or home. If the patient 1 1011 is at hospital then the computing unit 107 determines that the patient is not ready to be discharged from the hospital. If the patient 1 1011 is at home then the computing unit 107 provides appropriate suggestions to the patient to improve the recovery score. The computing unit 107 determines the parameter based on which the recovery score was less and provides the suggestion to improve the recovery score based on the parameter which has less recovery score.

In an exemplary embodiment, if the recovery score generated for the parameter, activity level is less, then the computing unit 107 provides the suggestions to increase activity level of the patient 1 1011 by certain number of activity units and thereby improve the recovery score. For example, if the activity level is less than 75% of the targeted activity level in the recovery plan for the patient 1 1011, the suggestion provided may be to walk around for 5 to 10 minutes 3 times a day. Similarly, if the body temperature of the patient 1 1011 at home is more than the normal body temperature, then the computing unit 107 suggests the patient 1 1011 to check with the physician for fever medication.

In some other embodiments, even after the patient 1 1011 is discharged from the hospital, the patient 1 1011 is continuously monitored to provide appropriate suggestions to the patient 1 1011 to cure the illness.

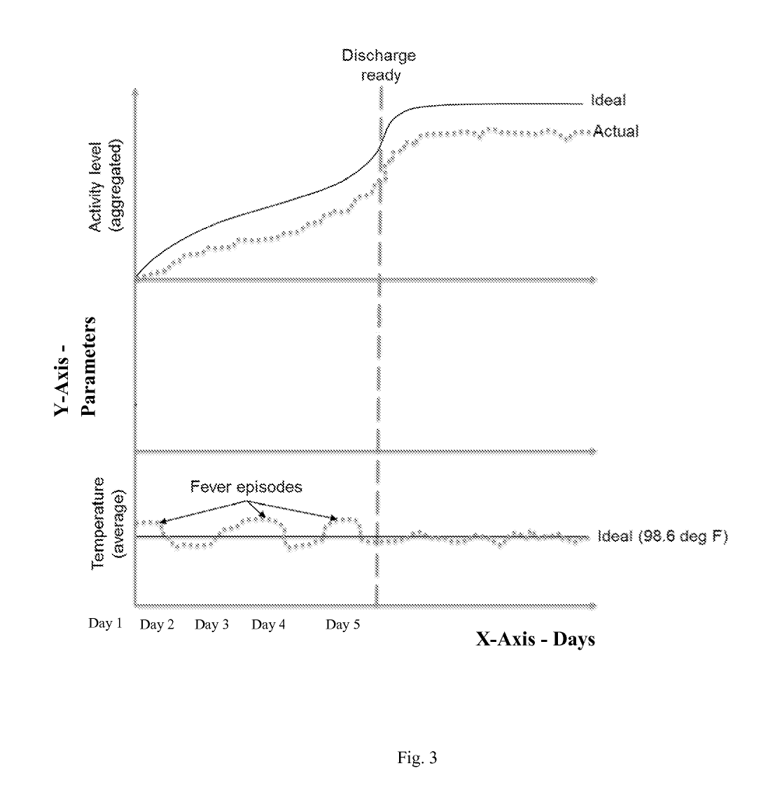

FIG. 3 illustrates graphical representation of recovery of patient on the display unit 113 in accordance with an exemplary embodiment of the present disclosure. In the above illustrated example (FIG. 2), the patient is monitored continuously for 5 days. The x-axis represents the no of days and the y-axis represents the physiological parameters monitored to determine discharge readiness condition for the patient. The physiological parameters monitored are the body temperature of the patient and activity level of the patient. As seen from the graphical representation, during the initial days of observation i.e day 1, day 2 and day 3, fever still persists as the body temperature is more than the ideal temperature. On the 5 day, the body temperature of the patient has come down to the ideal body temperature and the activity level of the patient has reached the ideal activity level. Therefore, the patient is ready for discharge on the 5 day of observation.

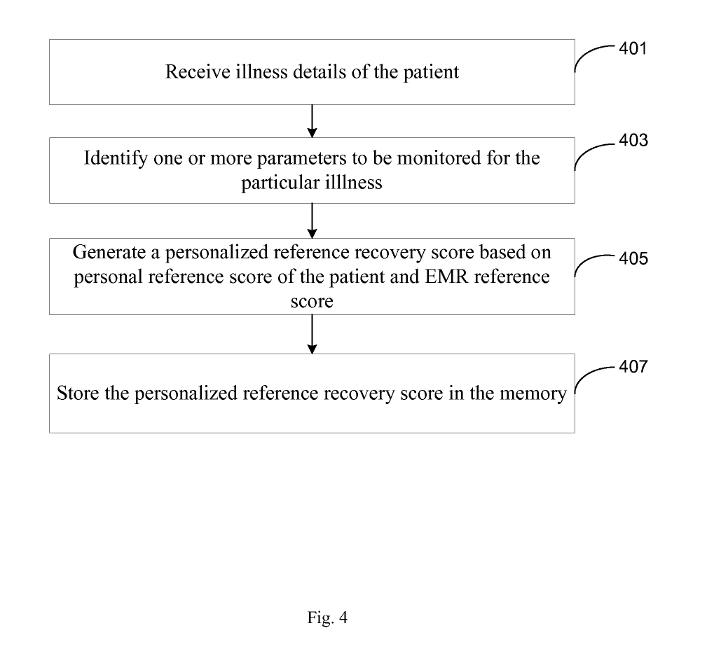

FIG. 4 shows a flowchart illustrating an exemplary process for determining reference recovery score of the patient.

As illustrated in FIG. 4, the method comprises one or more blocks for determining reference recovery score of the patient. The method may be described in the general context of computer executable instructions. Generally, computer executable instructions can include routines, programs, objects, components, data structures, procedures, modules, and functions, which perform particular functions or implement particular abstract data types. The order in which the method is described is not intended to be construed as a limitation, and any number of the described method blocks can be combined in any order to implement the method. Additionally, individual blocks may be deleted from the method without departing from the spirit and scope of the subject matter described herein. Furthermore, the method may be implemented in any suitable hardware, software, firmware, or combination thereof.

At block 401, the illness details are received from the patient. In one embodiment, the computing device 107 receives the illness details from the patient. The illness details may include the basic information from the patient about the illness. As an example, if the patient is suffering from fever, the basic information provided by the patient may include tiredness, feeling of cold, increased heart rate, shivering etc.

At block 403, the one or more parameters to be monitored for the particular illness are identified. In one embodiment, the computing device 107 identifies one or more parameters to be monitored for the identified illness. Based on the one or more parameters, the one or more sensors 103 are placed on the patient to monitor the health condition of the patient. The one or more sensors 103 monitor the physiological conditions of the patient and provide the one or more physiological data to the computing device 107. The patient also provides one or more non-physiological data to the computing device 107 through the mobile device 105. The processor 109 assigns a first weighted ratio to each of the physiological data and the non-physiological data to generate a weighted physiological data and a weighted non-physiological data. The computing device 107 generates a recovery score based on the weighted physiological data and the weighted non-physiological data.

At block 405, a personalized reference score for the patient is generated based on personal reference score of the patient and the EMR reference score. In one embodiment, the computing device 107 generates a personalized reference score for the patient. The personalized reference score is determined using the weighted personal reference score of the patient and the weighted EMR reference score. The weighted EMR reference score is determined by assigning a second weighted ratio to EMR reference score. The weighted personal reference score of the patient is determined by assigning a third weighted ratio to each of the physiological data and the non-physiological data recorded during normal health condition of the patient using one of one or more sensors 103 placed on the patient and one or more previous health report of the patient.

At block 407, the personalized reference score is stored in the memory. In one embodiment, the personalized reference score is stored in the memory for future use by the physician. The computing unit 107 compares the generated recovery score with the reference recovery score in order to determine whether the patient is ready for discharge from the hospital.

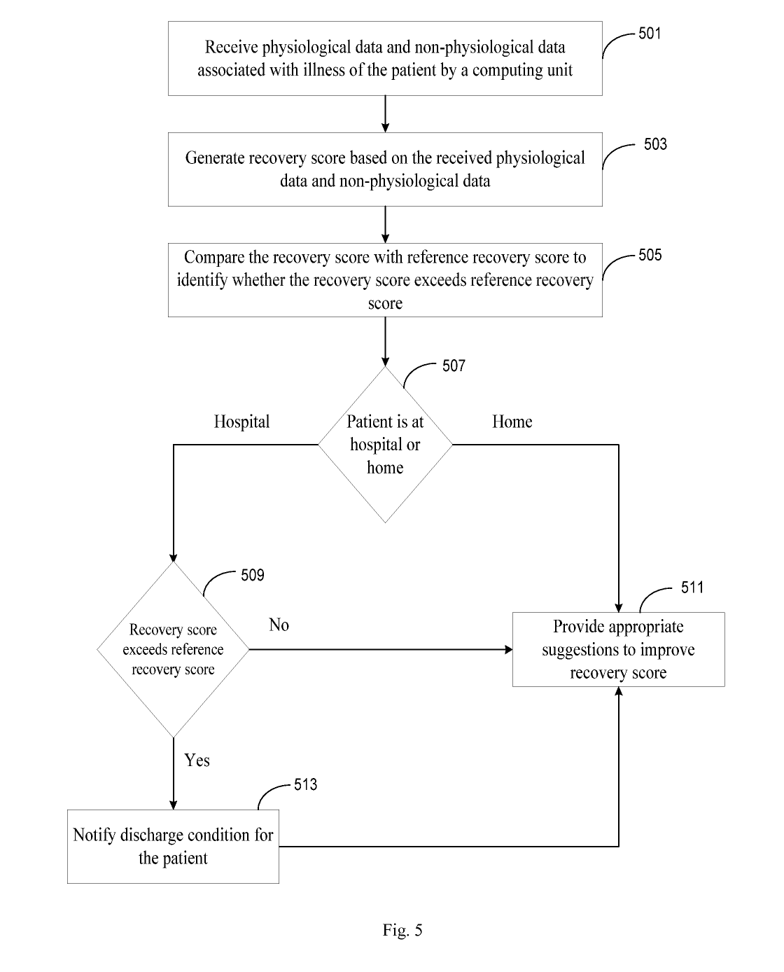

FIG. 5 shows a flowchart illustrating a method for determining discharge readiness condition for the patient.

As illustrated in FIG. 5, the method comprises one or more blocks for determining discharge readiness condition for the patient. The method may be described in the general context of computer executable instructions. Generally, computer executable instructions can include routines, programs, objects, components, data structures, procedures, modules, and functions, which perform particular functions or implement particular abstract data types. The order in which the method is described is not intended to be construed as a limitation, and any number of the described method blocks can be combined in any order to implement the method. Additionally, individual blocks may be deleted from the method without departing from the spirit and scope of the subject matter described herein. Furthermore, the method may be implemented in any suitable hardware, software, firmware, or combination thereof.

At block 501, the physiological data and non-physiological data associated with illness of the patient is received by the computing device 107. In one embodiment, the computing device 107 receives at least one of one or more physiological data from the plurality of sensors 103 placed on the patient and one or more non-physiological data from a mobile device 105 associated with the patient. The physiological data includes, but not limited to, temperature data, blood pressure data, activity data, electrocardiography (ECG) data and respiratory data and the non-physiological data includes, but not limited to, medicine intake data, food intake data and discomfort level data.

At block 503, a recovery score is generated by the processor 109 based on the received physiological data and the non-physiological data. In one embodiment, the processor 109 assigns a weighted ratio to each of the physiological data received from the plurality of sensors and the non-physiological data received from the mobile device to generate a recovery score.

At block 505, the generated recovery score is compared with the reference recovery score to identify whether the patient is ready for discharge or not.

At block 507, the computing device 107 determines whether the patient is at hospital or at home. If the patient is at hospital, then the method proceeds to block 509. Upon determining that the patient is at home, the method proceeds to block 511.

At block 509, the recovery score is compared with the reference recovery score. In one embodiment, a determination is made as to whether the recovery score exceeds the reference recovery score. If the recovery score exceeds the reference recovery score then the method proceeds to block 513 via “Yes”. If the recovery score is less than the reference recovery score then the method proceeds to block 511 via “No”.

At block 511, appropriate suggestions are provided to the patient to improve the recovery score. In one embodiment, upon determining the recovery score to be less than the reference recovery score, the computing unit 107 provides appropriate suggestions to the patient to improve the recovery score.

At block 513, a discharge notification is provided for the patient. In one embodiment, upon determining that the recovery score exceeds the reference recovery score, the computing unit provides a notification for discharge of the patient from the hospital.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor and a processor capable of processing and executing the queries. A non-transitory computer readable medium may comprise media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. The non-transitory computer-readable media comprise all computer-readable media except for a transitory. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as an optical fiber, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory computer readable medium at the receiving and transmitting stations or devices. An “article of manufacture” comprises non-transitory computer readable medium, hardware logic, and/or transmission signals in which code may be implemented. A device in which the code implementing the described embodiments of operations is encoded may comprise a computer readable medium or hardware logic. Of course, those skilled in the art will recognize that many modifications may be made to this configuration without departing from the scope of the invention, and that the article of manufacture may comprise suitable information bearing medium known in the art.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. On the contrary a variety of optional components are described to illustrate the wide variety of possible embodiments of the invention.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIGS. 4 & 5 show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processor or by distributed processing units.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims hereinafter appended.

Additionally, advantages of present disclosure are illustrated herein.

The present disclosure provides a method of determining discharge readiness condition of the patient wherein the recovery score is generated by continuously monitoring the health condition of the patient. Thereafter, a reference recovery score is personalized for each patient and the reference recovery score is not just based on EMR reference but also on the personal reference score of the patient which was recorded when the patient was healthy.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed:

1. A method for determining discharge readiness condition of a patient, the method comprising:
receiving, by a health monitoring computing device, at least one of one or more physiological data from plurality of sensors placed on the patient and one or more non-physiological data from a mobile device associated to the patient;
assigning, by the health monitoring computing device, a first weighted ratio to each of the physiological data received from the plurality of sensors and the non-physiological data received from the mobile device to generate a weighted physiological data and a weighted non-physiological data;
generating, by the health monitoring computing device, a recovery score using the weighted physiological data and the weighted non-physiological data; and
determining, by the health monitoring computing device, the discharge readiness condition of the patient when the recovery score exceeds a reference recovery score, wherein the reference recovery score is generated using a weighted personal reference score of the patient and a weighted Electronic Medical Record (EMR) reference score.

2. The method as claimed in claim 1, wherein the physiological data is at least one of body temperature data, blood pressure data, activity data, electrocardiography (ECG) data and respiratory data and the non-physiological data is at least one of medicine intake data, food intake data and discomfort level data.

3. The method as claimed in claim 1, wherein the sensors are at least one of a motion sensor, emotion sensor, physiological sensor and activity sensor.

4. The method as claimed in claim 1 further comprising displaying, by the health monitoring computing device, the recovery score on a display unit associated to the computing unit.

5. The method as claimed in claim 1, wherein the weighted EMR reference score is retrieved from an EMR database associated to the computing unit.

6. The method as claimed in claim 1, wherein the weighted EMR reference score is determined, by the health monitoring computing device, by assigning a second weighted ratio to an EMR reference score stored in the EMR database.

7. The method as claimed in claim 1, wherein the weighted personal reference score of the patient is determined, by the health monitoring computing device, by assigning a third weighted ratio to each of the physiological data and the non-physiological data recorded during normal health condition of the patient using one of one or more sensors placed on the patient and one or more previous health reports of the patient.

8. The method as claimed in claim 1 further comprising providing, by the health monitoring computing device, suggestions to the patient based on the recovery score.

9. A health monitoring computing device comprising:
a processor;
a memory, wherein the memory coupled to the processor which are configured to execute programmed instructions stored in the memory comprising:
receiving at least one of one or more physiological data from plurality of sensors placed on the patient and one or more non-physiological data from a mobile device associated to the patient;
assigning a first weighted ratio to each of the physiological data received from the plurality of sensors and the non-physiological data received from the mobile device to generate a weighted physiological data and a weighted non-physiological data;
generating a recovery score using the weighted physiological data and the weighted non-physiological data; and
determining the discharge readiness condition of the patient when the recovery score exceeds a reference recovery score, wherein the reference recovery score is generated using a weighted personal reference score of the patient and a weighted Electronic Medical Record (EMR) reference score.

10. The device of claim 9, wherein the sensors are at least one of a motion sensor, emotion sensor, physiological sensor and activity sensor.

11. The device of claim 9, wherein the instructions further configure the at least one processor to display the recovery score on a display unit associated to the computing unit.

12. The device of claim 9, wherein the instructions further configure the at least one processor to determine the weighted EMR reference score by assigning a second weighted ratio to an EMR reference score.

13. The device of claim 9, wherein the instructions further configure the at least one processor to determine the weighted personal reference score of the patient by assigning a third weighted ratio to each of the physiological data and the non-physiological data recorded during normal health condition of the patient using one of one or more sensors placed on the patient and one or more previous health reports of the patient.

14. The device of claim 9, further comprising providing suggestions to the patient based on the recovery score.

15. The device of claim 9, wherein the physiological data is at least one of body temperature data, blood pressure data, activity data, electrocardiography (ECG) data and respiratory data and the one or more non-physiological data is at least one of medicine intake data, food intake data and discomfort level data.

16. A non-transitory computer readable medium having stored thereon instructions for determining discharge readiness condition of a patient comprising machine executable code which when executed by at least one processor, causes the processor to perform steps comprising:
receiving at least one of one or more physiological data from plurality of sensors placed on the patient and one or more non-physiological data from a mobile device associated to the patient;
assigning a first weighted ratio to each of the physiological data received from the plurality of sensors and the one or more non-physiological data received from the mobile device to generate a weighted physiological data and a weighted non-physiological data;
generating a recovery score using the weighted physiological data and the weighted non-physiological data; and
determining the discharge readiness condition of the patient when the recovery score exceeds a reference recovery score, wherein the reference recovery score is generated using a weighted personal reference score of the patient and a weighted Electronic Medical Record (EMR) reference score.

17. The medium of claim 16, wherein the instructions further cause the at least one processor to determine the weighted EMR reference score by assigning a second weighted ratio to an EMR reference score.

18. The medium of claim 16, wherein the instructions further cause the at least one processor to determine the weighted personal reference score of the patient by assigning a third weighted ratio to each of the physiological data and the non-physiological data recorded during normal health condition of the patient using one of one or more sensors placed on the patient and one or more previous health reports of the patient.

19. The medium of claim 16, wherein the instructions further cause the at least one processor to provide suggestions to the patient based on the recovery score.

20. The medium of claim 16, wherein the instructions further cause the at least one processor to retrieve the weighted EMR reference score from an EMR database associated to the computing unit.
